(19) United States
(12) Patent Application Publication
(10) Pub. No.: US 2012/0214764 A1
(43) Pub. Date: Aug. 23, 2012

(54) METHOD AND KIT FOR GASTRO-INTESTINAL CLEANSING

(76) Inventor: Jose Rodriguez-San Juan, McAllen, TX (US)

(21) Appl. No.: 13/502,845

(22) PCT Filed: Oct. 19, 2010
(86) PCT No.: PCT/US2010/053191

§ 371 Date: Apr. 19, 2012

Related U.S. Application Data
(60) Provisional application No. 61/252,979, filed on Oct. 19, 2009.

(51) Int. Cl. A61K 31/695; A61P 1/00; A61K 31/19

(52) U.S. Cl. 514/63; 514/574

(57) ABSTRACT

A method of cleansing a mammalian GI tract is described and includes ingestion of polyethylene glycol (PEG) and magnesium citrate in a split-dose regimen. The split-dose regimen provides effective cleansing of the GI tract while minimizing discomfort and side effects to the patient A kit packaged with a first and second portion of PEG, and a first and second portion of magnesium citrate, and optionally simethicone is also provided.

### RELATED APPLICATION DATA

This application claims priority to U.S. provisional application 60/252,979 entitled “Method and Kit for Gastro-Intestinal Cleaning” filed Oct. 19, 2009, which is hereby entirely incorporated herein by reference.

### BACKGROUND

Various health procedures, such as colonoscopy, may require prior clearance of the gastro-intestinal (GI) tract. For example, a colonoscopy may involve insertion of an endoscope into the colon for detecting and imaging structural features, such as polyps or lesions. Cleansing the colon of residual matter may better allow detection and imaging of the colon tissue. Other procedures, such as radiological imaging or other diagnostics, and surgeries, may be better accomplished upon a cleared GI tract. Procedures for clearing the GI tract may also be useful for treatment of constipation and may be performed either in a healthcare facility or in a home setting.

Clearing the GI tract of matter may involve a combination of fasting, drinking certain liquids, and using laxatives. Some prior GI tract preparation compounds have contained relatively high amounts of phosphates. Oral ingestion of phosphates may raise osmotic pressure in the intestines, thus softening the matter. However, the side effects of phosphate ingestion include abdominal bloating and pain. In more severe cases, phosphate ingestion may cause kidney damage, such as by formation of phosphate salts in the renal tubes.

Other cleansing methods, such as lavage, require ingestion of a large quantity of fluids over a relatively short amount of time. Such methods may cause considerable discomfort and inconvenience for those using such methods.

Accordingly, there is a need for a method and kit of compounds for clearing the GI tract that minimizes patient discomfort and operates without substantial adverse side effects.

### SUMMARY

A method of cleansing a mammalian GI tract may include ingestion of polyethylene glycol (PEG) and magnesium citrate in a split-dose regimen. A first portion of PEG may be initially ingested. Thereafter, a first portion of magnesium citrate may be ingested. A second portion of PEG may then be ingested. Thereafter, a second portion of magnesium citrate may be ingested. In some embodiments, ingestion of simethicone may accompany ingestion of the second portion of magnesium citrate.

A kit may include a first and second portion of PEG, and a first and second portion of magnesium citrate. In some embodiments, a kit may include a portion of simethicone.

### BRIEF DESCRIPTION OF THE FIGURES

FIG. 1 illustrates one embodiment of a method of cleansing the GI tract.

FIG. 2 illustrates a timeline for the embodiment of FIG. 1.

### DETAILED DESCRIPTION

A method of cleansing a GI tract may comprise a mammal, such as a person or animal, ingesting various quantities of various compounds at various times prior to a health procedure. In some embodiments, methods of GI tract cleansing may comprise ingestion of polyethylene glycol (PEG), magnesium citrate and simethicone as described herein. Some embodiments may involve ingestion in a split-dose regimen of more than one portion of a compound. The timing of such portions may improve the efficiency of GI tract cleansing and the palatability of ingestion for a patient.

PEG and magnesium citrate may cause the intestines to retain water, thus causing residual matter to soften and promoting peristalsis.

Simethicone may comprise a mixture of polydimethylsiloxane and silica gel. Simethicone may alter the surface energy of gas bubbles in the digestive system, thus increasing the rate at which gases exit the intestines.

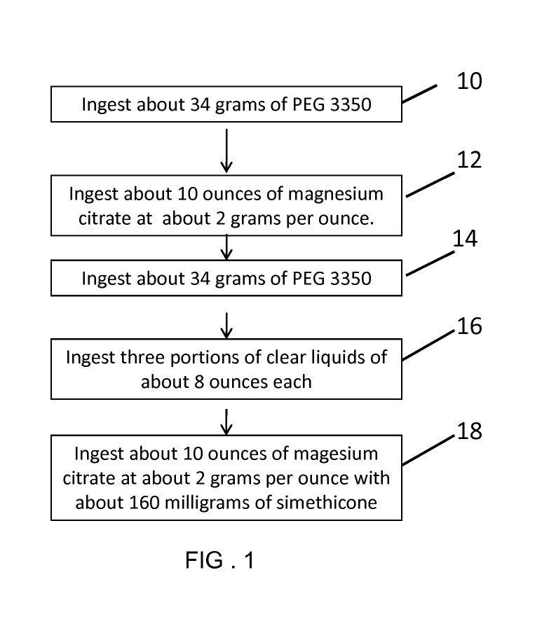

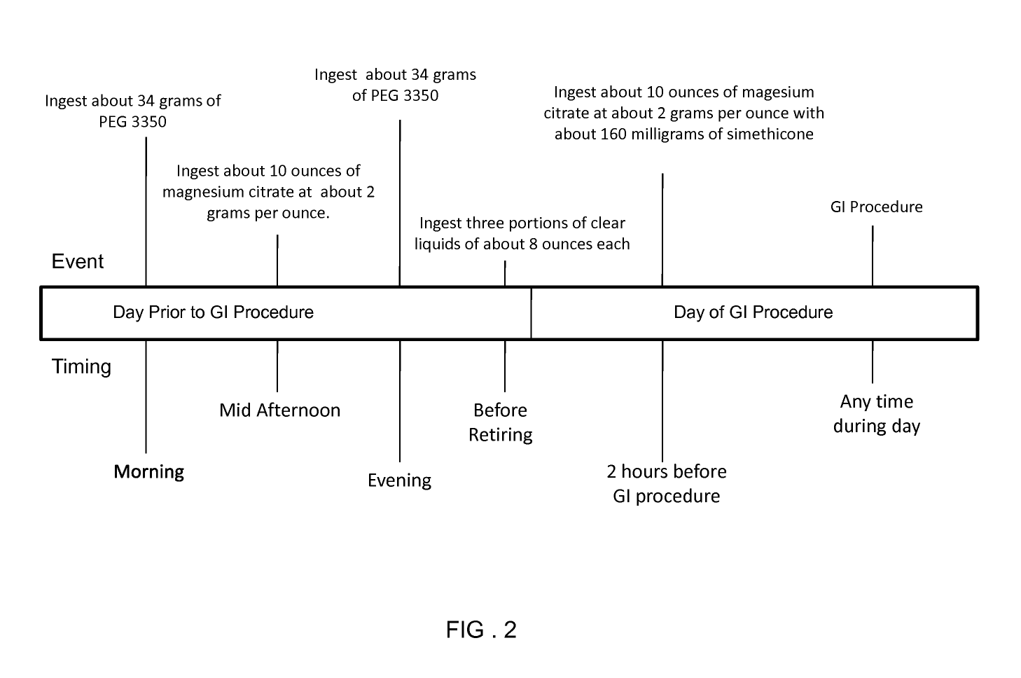

As may be seen from FIGS. 1 and 2, in step 10, in one embodiment, a first portion of about 34 grams of PEG 3350 (polyethylene glycol having an average molecular weight of 3350 grams/mole) may be ingested in the morning (e.g., at about 9:00 a.m.) of the day prior to the procedure. PEG 3350 may be provided in powdered form, and may be more comfortably ingested if first dissolved in water and ingested as a drink. In this embodiment the polyethylene glycol may be dissolved in about 16 ounces of water. Other quantities of PEG 3350 in the range of about 10 to about 50 grams may also be effective. The specific amount of PEG used may in some embodiments depend upon body weight. Also, PEG having other average molecular weights, such as 8000 grams/mole, may be ingested in suitable amounts to promote an increase in osmotic pressure within the intestines. In general, the amount of PEG necessary to accomplish GI tract cleansing may range from about 10 grams to about 100 grams. A suitable dose may depend on the osmolarity of PEG, and, at least for higher molecular weight PEG compositions, may in part be determined by the effectiveness of a PEG compound in lubricating the GI tract.

In step 12, in approximately mid-afternoon (e.g., at about 4:00 p.m.) of the day prior to the procedure, a first portion of about 10 ounces of magnesium citrate may be ingested. The magnesium citrate may be provided in liquid form at a concentration or about 2 grams of magnesium citrate per fluid ounce: for ready ingestion. Thus, about 10 ounces, may provide about 20 grams of magnesium citrate. Other quantities of magnesium citrate <in the range of about 1 to about 3 grams per ounce may also be effective.

Further, other concentrations of magnesium citrate may be used at other suitable volumes so as to provide substantially the same amount of magnesium citrate. However, concentrations higher than about 2 grams per fluid ounce may taste salty. Magnesium titrate solutions at those concentrations may generally be unpleasant to ingest and have a bitter taste. In some embodiments magnesium citrate may be ingested along with a flavoring agent that preferably is not colored red or purple. When ingested with a flavoring agent, magnesium citrate may be palatable at a concentration above about 2 grams per fluid ounce. In some embodiments other magnesium salts, including but not limited to magnesium oxide, may be used. Such salts may be effective to cause the intestines to retain water when used at a concentration and volume sufficient to provide between about 1 to about 3 grams of magnesium.

In step 14, in approximately the evening (e.g., at about 8:00 p.m.) of the day prior to the procedure, a second portion of about 34 grams of PEG 3350 may be ingested. The time between the first ingestion of PEG in step 10 and the second ingestion of PEG in step 14 may be about 11-12 hours. In some embodiments a time delay between a first ingestion of PEG and a second ingestion of PEG may be between about 9 hours and about 13 hours. As in step 10, the PEG 3350 may be dissolved in about 16 ounces of water to aid ingestion. Also as in step 14, other quantities of PEG 3350 in the range of about 10 to about 50 grams may also be effective, and PEG having other average molecular weights, such as 8000 grams/mole, may be ingested in suitable quantities. The first and second portions of PEG do not have to be the same quantity, or of the same PEG concentration or molecular weight.

In step 16, approximately close to or just before retiring for the day prior to the procedure, one or more about 8-ounce portions of a substantially clear liquid, such as water, may be ingested. The ingestion of those portions of substantially clear liquid may be useful to assist an individual to maintain proper hydration. In one embodiment, three about 8-ounce portions of a substantially clear liquid may be ingested. Such ingestion is however not essential for cleansing the GI tract, and in some embodiments the ingestion of those liquids may be optional.

In step 18, at least approximately two hours prior to the procedure, a second portion of about 10 ounces Of magnesium citrate solution at a concentration of about 2 grams per ounce may be ingested, along with a portion of about 160 milligrams of simethicone. While it may generally be desirable to ingest simethicone along with magnesium citrate in step 18, some methods may involve the ingestion of magnesium citrate without simethicone. As in step 12, the magnesium citrate may be provided in liquid form for ready ingestion, and in suitable volumes and concentrations. Simethicone may be provided in 80-mg tablets, and two of such tablets may satisfy the 160-mg dose of simethicone in this step. Other quantities of simethicone in a range from about zero up to about 320 milligrams may also be effective.

During the method of FIG. 1, only substantially clear liquids only may be ingested up to about two hours before the procedure. Solid foods, milk and milk products may not be ingested during performance of the method. Substantially clear liquids may comprise strained fruit juice without pulp, such as apple juice, white grape juice and lemonade; water; clear broth or bouillon; coffee (without milk, cream or non-dairy creamer); tea; sports drinks such as Gatorade; soft drinks (whether or not carbonated); and flavored drinks such as Kool-Aid. Substantially clear liquids may also comprise substances that become solids-free liquid at or shortly after ingestion, such as ice popsicles and gelatinous desserts such as the material sold under the trademark Jell-O by Krafts Foods Holdings Inc. In one embodiment, substantially clear liquids may not comprise red- or purple-colored drinks or other substances. Other substances may be suitable for ingestion, and should be readily dispersible into a liquid form in the GI tract, and preferably should not include pulp or other colloidal particles. Such substances preferably should be a color other than red or purple, and should not interfere with inspection or imaging of the surface of the GI tract.

PEG 3350 and other suitable PEG compounds maybe provided from a variety of sources and in a variety of forms. Those PEG compounds may be provided as a solid tablet or powder may be provided as a solution suitable, for direct ingestion, or may be provided as a concentrated solution that may be diluted to the desired concentration by a patient. It may be convenient to provide a first portion of PEG and a second portion of PEG in the same form, however this is not essential. For example, PEG 3350 may be commercially available under the trade names SoftLax, MiraLax and GlycoLax. Other PEG compounds may be commercially available under the trade names GoLYTELY, NuLytely, Fortrans, TriLyte, Colyte, Halflytely, and MoviPrep.

Magnesium citrate or other suitable magnesium salts may be provided from a variety of sources and in a variety of forms. Those magnesium salts may be provided as a solid tablet or powder, may be provided as a solution suitable for dire& ingestion, or maybe provided as a concentrated solution that may be diluted to the desired concentration by a patient. It may be convenient to provide a first portion of magnesium salt and a second portion of magnesium salt in the same form, however this is not essential. For example, magnesium citrate may be commercially available under the trade names Citromag or Citroma, may be provided in pill form, and may be provided in a variety of concentrations.

Simethicone may be provided from a variety of sources and in a variety of forms. For example, magnesium citrate may be commercially available under the trade names GasAid, Gas-X, Imodium, Infacon, Maalox, Mylanta, Mylicon, and Rolaids, among others. Simethicone may also be provided in capsule, chewable tablet, suspension and dissolving membrane form.

The compounds used in the foregoing method embodiments may be provided in a kit form. By way of example only, a kit may comprise two 10-oz bottles of magnesium citrate, four 8-oz bottles of Miralax PEG 3350, and two 80-mg simethicone tablets. More generally, compounds as described above may be provided in a kit as solid tablets or powders, may be provided as solutions suitable for direct ingestion, or may be provided as concentrated solutions that may be diluted to the desired concentration by or for a patient. Instructions on performing the foregoing method embodiment may accompany the kit. Such a kit, or just the instructions, may be provided by a health-care provider, a pharmacy, a pharmaceutical compounds manufacturer, or other party connected with preparation of a patient for a health procedure, such as colonoscopy or surgery, or for other purposes that are better performed with a cleaned GI tract.

Instructions for the foregoing method may remind patients that performing the method may cause multiple bowel movements. The instructions may also advise patients to refrain from ingesting any aspirin, iron pills, and blood thinners such as Coumadin and Plavix at least five days before the procedure. The instructions may further advise diabetic patients that a regularly-scheduled dose of insulin may be administered in the days before the procedure, and that half of a regularly-scheduled dose of insulin may be administered on the day of the procedure.

The foregoing method and kit for clearing a GI tract may be performed either in a healthcare facility or in a home setting, or at any other suitable location(s).

While many examples in this document refer to methods or kits of gastro-intestinal cleaning, it is understood that those methods and kits are described in an exemplary manner only and that other methods may be used. Additionally, other ingredients may be used, depending on the particular needs. Although the foregoing specific details describe certain embodiments, persons of ordinary skill in the art will recognize that various changes may be made in the details of these embodiments without departing from the spirit and scope of this invention as defined in the appended claims and considering the doctrine of equivalents. Therefore, it should be understood that this invention is not limited to the specific details shown and described herein.

1. A method of cleansing the GI tract comprising:
ingesting a first portion of polyethylene glycol, the first portion of polyethylene glycol in an amount in the range of about 10 grams to about 50 grams;
after ingesting said first portion of polyethylene glycol, ingesting a first portion of magnesium citrate, the first portion of magnesium citrate in an amount in the range of about 10 grams to about 30 grams;
after ingesting said first portion of magnesium citrate, ingesting a second portion of polyethylene glycol, the second portion of polyethylene glycol in an amount in the range of about 10 grams to about 50 grams; and
after ingesting said second portion of polyethylene glycol, ingesting a second portion of magnesium citrate, the second portion of magnesium citrate measuring from about 10 grams to about 30 grams.

2. The method of claim 1, wherein said first portion of polyethylene glycol and said second portion of polyethylene glycol are each provided in the form of one or more tablets, a powder, or a liquid solution.

3. The method of claim 1, wherein said first portion of magnesium citrate and said second portion of magnesium citrate are each provided in the form of one or more tablets, a powder, or a liquid solution.

4. The method of claim 1, wherein said first portion of magnesium citrate and said second portion of magnesium citrate are provided as a stock portion that may be divided to form said first portion of magnesium citrate and said second portion of magnesium citrate.

5. The method of claim 1, wherein said first portion of polyethylene glycol and said second portion of polyethylene glycol are provided as a stock portion that may be divided to form said first portion of polyethylene glycol and said second portion of polyethylene glycol.

6. The method of claim 1, wherein said ingestion of the first portion of polyethylene glycol occurs between about 9 hours and about 13 hours before said ingestion of the second portion of polyethylene glycol.

7. The method of claim 1, further comprising ingesting at least three substantially 8-fluid-ounce portions of one or more substantially clear liquids.

8. The method of claim 1, further comprising ingestion of simethicone at a mass up to about 320 milligrams and wherein the simethicone is ingested along with the second portion of magnesium citrate.

9-15. (canceled)

16. A pharmaceutical composition comprising:
a first portion of polyethylene glycol, the first portion of polyethylene glycol in an amount of about 34 grams;
a first portion of magnesium citrate, the first portion of magnesium citrate in an amount of about 20 grams;
a second portion of polyethylene glycol, the second portion of polyethylene glycol in an amount of about 34 grams; and
a second portion of magnesium citrate, the second portion of magnesium citrate in an amount of about 20 grams.

17. The pharmaceutical composition of claim 16, wherein said first portion of polyethylene glycol and said second portion of polyethylene glycol are each provided in the form of one or more tablets, a powder, or a liquid solution.

18. The pharmaceutical composition of claim 16, wherein said first portion of magnesium citrate and said second portion of magnesium citrate are each provided in the form of one or more tablets, a powder, or a liquid solution.

19. The pharmaceutical composition of claim 18 wherein said first portion of magnesium citrate and said second portion of magnesium citrate are each provided in the form of a liquid solution wherein said liquid solution contains a flavoring agent.

20-22. (canceled)

23. A pharmaceutical composition comprising:
a first portion of polyethylene glycol, the first portion of polyethylene glycol in an amount of about 10 grams to about 50 grams;
a first portion of magnesium citrate, the first portion of magnesium citrate in an amount of about 10 to about 30 grams;
a second portion of polyethylene glycol, the second portion of polyethylene glycol in an amount of about 10 to about 50 grams; and
a second portion of magnesium citrate, the second portion of magnesium citrate in an amount of about 10 to about 30 grams.

24. The pharmaceutical composition of claim 23, wherein said first portion of polyethylene glycol and said second portion of polyethylene glycol are each provided in the form of a tablet, a powder, or a liquid solution.

25. The pharmaceutical composition of claim 23, wherein said first portion of magnesium citrate and said second portion of magnesium citrate are each provided in the form of a tablet, a powder, or a liquid solution.

26. The pharmaceutical composition of claim 25 wherein said first portion of magnesium citrate and said second portion of magnesium citrate are each provided in the form of a liquid solution wherein said liquid solution contains a flavoring agent.

27. The pharmaceutical composition of claim 23 wherein said first portion of magnesium citrate and said second portion of magnesium citrate are each provided in the form of a solid tablet or powder and further comprising a flavoring agent that may be added to said first portion and second portion of magnesium citrate.

28. The pharmaceutical composition of claim 23 further comprising instructions for using said article by ingesting said first portion of polyethylene glycol, said first portion of magnesium citrate, said second portion of polyethylene glycol and said second portion of magnesium citrate at specific time intervals.

29. The pharmaceutical composition of claim 28 further comprising instructions for how to partition said first portion of polyethylene glycol and said second portion of polyethylene glycol from a stock portion of polyethylene glycol and/or how to partition said first portion of magnesium citrate and said second portion of magnesium citrate from a stock portion of magnesium citrate.

30. The pharmaceutical composition of claim 23 further comprising up to about 320 milligrams of simethicone.
